(19) United States
(12) Patent Application Publication
(10) Pub. No.: US 2026/0083902 A1
(43) Pub. Date: Mar. 26, 2026

(54) DEVICE FOR COUPLING A SYRINGE TO AN ACTUATOR

(71) Applicant: TRASIS S.A., ANS (BE)

(72) Inventors: Jean-Luc MORELLE, LIEGE (BE); Philippe DUMONT, JUPILLE-SUR-MEUSE (BE); Philippe BEKAERT, HUY (BE)

(21) Appl. No.: 19/112,626

(22) PCT Filed: Sep. 5, 2023
(86) PCT No.: PCT/EP2023/074306

§ 371 Date: Mar. 18, 2025

(30) Foreign Application Priority Data
Sep. 21, 2022 (EP) 22196981.9

(51) Int. Cl. A61M 5/00; A61M 5/168; A61M 5/20; A61M 5/315

(52) U.S. Cl. CPC A61M 5/007; A61M 5/16813; A61M 5/2033; A61M 5/3158; A61M 2205/332; A61M 2205/702

(57) ABSTRACT

A system for reversibly coupling a syringe for injecting a calibrated product to an actuator, including the actuator, the syringe provided with a pusher having a pusher button and a coupling device, the coupling device including an internal body with longitudinal axis and an outer sleeve intended to move with respect to the internal body along the longitudinal axis, the internal body including a sliding pin ending with a play-take-up plate that is held longitudinally inside the internal body in a rest position by a prestressed spring as well as at least one pair of hooks arranged longitudinally inside the internal body, mounted pivoting and retractable two by two with respect to two respective pins perpendicular to the longitudinal axis, an external part of the hooks being located opposite an internal part of the sleeve, the hooks of a same pair each having a zone for hooking.

### CROSS-REFERENCE TO PRIOR APPLICATIONS

This application is a U.S. National Phase application under 35 U.S. C. § 371 of International Application No. PCT/EP2023/074306, filed on Sep. 5, 2023, and claims benefit to European Patent Application No. EP 22196981.9, filed on Sep. 21, 2022. The International Application was published in French on Mar. 28, 2024 as WO/2024/061619 under PCT Article 21(2).

### FIELD

The present invention relates to the technical field of automated devices for the administration of precise doses of drugs, e.g. intravenously, subcutaneously, etc., to a patient, more particularly doses of radiopharmaceutical products.

More particularly, the invention relates to a system for coupling a syringe to an actuator.

### BACKGROUND

Automated devices for the administration of radiopharmaceutical doses are known, which, from a multidose vial, collect a volume of solution corresponding to a requested quantity of radioactivity. The device then pushes the collected volume back into a transfer line connected to the patient. The assembly of lines and other devices for the administration of doses to the patient is a kit that must be replaced daily.

In a particular embodiment where dosing is achieved by means of a syringe combined with a three-way valve, an example of such a kit is represented in the diagram shown in FIG. 1. The kit 100 comprises:

a syringe 101 with a nominal capacity of e.g. 10 ml;

a vial 107 of radiopharmaceutical product, e.g. with a maximum capacity of 20 ml, typically filled with 10-15 ml of solution, and fitted with a septum to be pierced;

a three-way valve 103;

a container with saline 109 and a line 108;

a collection line 106 connecting the radio-pharmaceutical product vial 107, via a needle 118 piercing the septum of the vial and a tube running through a bubble detector 112, to the three-way valve 103;

a “Tee” 104 connecting the three-way valve 103 to the NaCl solution line 108, to the injection line 110 and to the syringe 101;

the line 108 connecting the saline container 109 to the injection line 110 through a non-return valve 105;

the injection line 110 comprising a non-return valve 105′, a ventilated filter 111 and a self-closing Luer fitting 113. Said line passes through a bubble detector 112′ upstream of the self-closing fitting 113;

a patient extension set 114 connected downstream of the line 110 by means of a Luer fitting 115 and comprising at its other end, another Luer fitting 116, the connector 116 connecting either to a catheter, or to a transfusion line or else to another syringe for preparing a dose in a syringe that is external to the machine with the aim to manually administer the dose to a patient;

a dose calibrator 117 being located at the level of the syringe 101.

The automated sequence of operations achieved with the kit 100 is the following:

a specific quantity of solution is collected from the vial 107 by suction through the collection line 106 via the three-way valve 103 by the syringe 101 the plunger of which is pulled by the actuator, the collection line being opened and the injection line closed by the three-way valve 103;

the concentration of the injectable solution can be adjusted by collecting via the three-way valve, an appropriate quantity of saline from the saline container 109, via the line 108, which is sucked into the syringe 101;

the solution, diluted if appropriate, is injected via the three-way valve 103 through the injection line 110, the collection line 106 being closed. The ventilated filter 111 prevents the passage of bubbles to the patient and the detector 112′ checks the passage;

the solution is directed to the patient extension set 114.

A person skilled in the art who is in a position to design such a device must take into account various considerations which determine:

the required precision of collection: the dose delivered to the patient must be very precise (e.g. +/−5%), regardless of the concentration of the initial product. Both the concentration of the initial product and the dose to be administered (which depends on the weight of the patient and may even be a pediatric dose) are highly variable;

the choice of the maximum syringe volume; and the control of the pressure exerted on the syringe when it is actuated.

Other ergonomic and practical considerations determine the placement and replacement of the kit including the syringe.

According to such considerations, placing and replacing the kit on its actuator, called an injector, should be simple and quick. The operation and performance of the administration system have to be independent of the operator's dexterity.

More particularly, attaching and detaching the pusher of the syringe to the coupling of the actuator, once the kit is in place, has to be automatic. The coupling should not include any “active” components, i.e. that would need to be specifically activated by the control system beyond the syringe actuator as such. The mechanism for hooking and releasing the pusher button should thus be passive. Furthermore, when the pusher button is attached to the actuator coupling, no play can remain between the pusher button and the actuator.

Document WO 2018/075386 A1 discloses an engagement mechanism associated with a reversing plunger of a fluid injector configured to engage in a removable manner, an engagement portion at a proximal end of a rolling diaphragm syringe, having a flexible sidewall configured to roll on itself when touched by the plunger. The engagement mechanism comprises a plurality of engagement elements that are movable reversibly and radially relative to the syringe engagement portion between a first position, wherein the plurality of engagement elements are disengaged from the syringe engagement portion, and a second position, wherein the plurality of engagement elements are engaged with the syringe engagement portion. The engagement mechanism further comprises a drive mechanism for shifting the plurality of engagement elements between the first position and the second position.

Document WO 2015/085929 A1 discloses a syringe plunger clamping device for syringe pumps comprising a housing, a lever, a connecting rod, a limiting element, an elastic element, a clamping jaw and a button. The middle portion of the lever is rotatably connected to the housing, the connecting rod slidably extending through the housing, and one end of the connecting rod being movably connected to one end of the lever. The limiting element is attached to the connecting rod, the elastic element is sleeved onto the connecting rod, and two ends of the elastic element abut against the internal wall of the housing and the limiting element, respectively. The clamping jaw is arranged on the housing and is securely connected to the end of the connecting rod that is far from the lever, and the clamping jaw is engaged with the outer surface of the housing so as to clamp the plunger of a syringe. The button slides through the housing and is in contact with the end of the lever that is far from the connecting rod. The button can slide into the housing to push in the lever into rotation so as to cause the connecting rod to slide towards the outside of the housing.

Document WO 97/07838 A1 discloses a syringe driver intended to perform the controlled parenteral perfusion of a medical fluid using an available, disposable syringe. The driver device comprises a frame with a movable carriage, in which a force-applying element is housed mounted thereon. A disposable syringe is mounted on the frame and a long micro-bore tube is attached to the syringe outlet tip. When a force is applied to the plunger of the syringe, the fluid is expelled from the syringe, but its flow rate depends on the diameter of the micro-bore tube. The driver device allows to administer multiple, sequential doses of fluid.

Document EP 1 447 105 A2 discloses a liquid injector having a gripping detector arranged on the front face of a plunger pusher for detecting when a plunger is pushed by the plunger pusher and for detecting thereby when a plunger flange arranged on the plunger is gripped by a pair of engagement claws on the plunger pusher. The plunger pusher cannot be actuated when the plunger flange is not gripped by the engagement claws.

Document EP 1 779 830 A1 discloses a medication transfer device comprising a vertically movable vial clamp for holding vials of different sizes in position with their septum downwards, a syringe holder and an actuator which comprises a clamp for gripping the plunger of the syringe and a movable plate for supporting a bag containing a perfusion solution. The syringe holder may be moved between a suction position under the vial clamp with the syringe or needle attached thereto pointing toward the septum of the vial and an expulsion position in which the syringe or the needle attached thereto points toward an injection port of the perfusion bag.

When the vial and the syringe are in place with the syringe holder in the suction position, the vial clamp moves downwards so that the syringe needle pierces the septum. The actuator retracts the plunger to a predetermined distance, thereby sucking in the desired amount of medication. Then, the vial clamp moves up, thereby releasing the needle. Such event is followed by the movement of the syringe holder toward the expulsion position. The plate then moves toward the syringe holder until the needle enters the injection port. The actuator then pushes in the plunger, and the medication is expelled into the bag, whereupon the plate is moved away from the syringe to disengage the needle.

Document WO 2014/129493 A1 discloses a chemical solution suction device provided with a plunger holding mechanism that holds a plunger element, and a cylinder holding mechanism that holds a cylinder element. The plunger holding mechanism and the cylinder holding mechanism are designed to be movable so that the plunger holding mechanism and the cylinder holding mechanism move toward and away from each other. In addition, the chemical solution suction device is provided with a pressing mechanism that applies a pressing force to the plunger holding mechanism and/or to the cylinder holding mechanism, thereby moving one of the plunger holding mechanism and the cylinder holding mechanism from (i) a first state in which the plunger holding mechanism and the cylinder holding mechanism are located in the vicinity of each other to (ii) a second state in which the two mechanisms are separated from each other by a predetermined distance.

### SUMMARY

In an embodiment, the present invention provides a system for reversibly coupling a syringe for injecting a calibrated product to an actuator, comprising the actuator, the syringe provided with a pusher having a pusher button and a coupling device, the coupling device comprising an internal body with longitudinal axis and an outer sleeve intended to move with respect to the internal body along the longitudinal axis, the internal body comprising a sliding pin ending with a play-take-up plate that is held longitudinally inside the internal body in a rest position by a prestressed spring as well as at least one pair of hooks arranged longitudinally inside the internal body, mounted pivoting and retractable two by two with respect to two respective pins perpendicular to the longitudinal axis, an external part of the hooks being located opposite an internal part of the sleeve, the hooks of a same pair each having a zone for hooking to a proximal end of the play-take-up plate, and being connected to each other to a distal end by a return spring, wherein the distal part of the hooks is provided, outwards, with a bulge intended to be inserted into a hollow zone of a first depth of the internal profile of the sleeve in a rest position of the coupling device where the hooks are upright and in position for horizontally locking the pusher button with the play-take-up plate, wherein the relative sliding of the sleeve with respect to the internal body, wherein the sleeve moves away from the distal end of the hooks causes the hooks to open by pivoting about their respective pivoting pins, because the bulge of each hook penetrates in the direction opposite that of the motion of the sleeve, a hollow zone of a second depth of the internal profile of the sleeve, the hollow zone of a second depth being adjacent to the hollow zone of a first depth, the first depth being greater than the second depth, and wherein the region for hooking the hook is defined by a protruding portion with an inwardly sloping chamfer followed by a recess below the protruding portion, the recess being dimensioned so as to lock by means of the hooks, the play-take-up plate adjoining the pusher button of the syringe, the play-take-up plate exerting, thanks to the prestressed spring, a force directed toward the pusher button so as to eliminate any play between the pusher button and the coupling device.

### BRIEF DESCRIPTION OF THE DRAWINGS

The present invention will be described in even greater detail below based on the exemplary figures. The invention is not limited to the exemplary embodiments. Other features and advantages of various embodiments of the present invention will become apparent by reading the following detailed description with reference to the attached drawings which illustrate the following:

FIG. 1 schematically shows the components and operation of a kit used in an automatic device for administering a radiopharmaceutical dose in a particular embodiment of the invention.

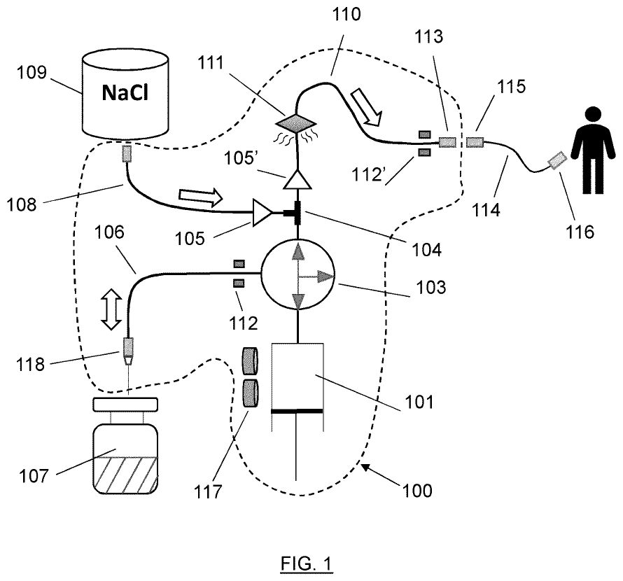

FIG. 2 represents a three-dimensional overall view of the actuator, syringe and device for coupling the two elements according to an embodiment of the present invention.

FIG. 3 shows different sectional views of the coupling and its components (in orthogonal planes).

FIG. 4 shows views of the coupling in positions with closed hooks (a) and open hooks (b), respectively.

FIG. 5 shows in a plurality of views the mechanism for securing the syringe with relative movement of the coupling with respect to the syringe and the opening/closing of the hooks.

FIG. 6 shows in a plurality views the mechanism for separating the syringe with relative movement of the coupling with respect to the syringe and the opening/closing of the hooks.

### DETAILED DESCRIPTION

In an embodiment, the present invention provides a simple, precise and rapid device for coupling radiopharmaceutical kits and an automated system for distributing a dose to a patient.

In an embodiment, the present invention provides a play-free coupling of the actuator with the pusher of the syringe.

A first aspect of the present invention relates to a device for the reversible coupling of a syringe to an actuator, comprising an internal body with longitudinal axis and an outer sleeve intended to move with respect to the internal body along the longitudinal axis, the internal body comprising a sliding pin ending with a play-take-up plate held longitudinally inside the internal body in a rest position by a prestressed spring as well as at least one pair of hooks arranged longitudinally inside the internal body, mounted pivoting and retractable two by two around two respective pins that are perpendicular to the longitudinal axis, an external part of the hooks being located opposite an internal part of the sleeve characterized in that the hooks of a same pair each have a zone for hooking to a proximal end of the play-take-up plate, and are connected to each other at a distal end by a return spring, the distal part of the hooks being provided, outwards, with a bulge intended to be inserted into a hollow zone of a first depth of the internal profile of the sleeve in a rest position of the coupling device where the zone for hooking the hooks is in contact position for locking the pusher button, the hooks being closer, the relative sliding of the sleeve with respect to the internal body, wherein the sleeve moves away from said distal end of the hooks (upwards, if the syringe is arranged above the coupling device), causing the hooks to open by pivoting about the respective pivoting pins, because the bulge of each hook penetrates into a hollow zone of a second depth of the internal profile of the sleeve, the hollow zone of a second depth being adjacent to the hollow zone of a first depth, the first depth being greater than the second depth.

According to preferred embodiments, the device further comprises at least one of the following features or an appropriate combination of a plurality thereof:

the device comprises 2 to 4 pairs of hooks;

the hooks have a hooking region defined by a protruding portion with an inwardly sloping chamfer followed by a recess below the protruding portion, the recess being dimensioned to lock without any play by means of the hooks, the play-take-up plate pushed by its prestressed spring and attached to a pusher button of a syringe.

A second aspect of the invention relates to a reversible coupling of a syringe for injecting calibrated product and of an actuator, said syringe being provided with a pusher with a pusher button, said actuator being intended to be coupled to the pusher of the syringe by means of the device described above, characterized in that, in the coupled position, the pusher button of the syringe is locked in the recess between the protruding part of the hooks (44) and the play-take-up plate which, by means of the prestressed spring, exerts a force directed toward the pusher so as to eliminate any play between the pusher and the coupling device.

Advantageously, the reversible coupling comprises a force sensor arranged behind the coupling mechanism between the actuator and the coupling device and is intended to measure the forces directly during the injection and during the coupling.

A third aspect of the invention relates to an automatic machine for the administration of a dose of product, comprising an actuator (3) and at least one syringe (5) for the injection of a calibrated product, provided with a pusher (6) with a pusher button (7), and a reversible coupling of the syringe (5) and of the actuator (3).

Preferably, the automatic machine for the administration of a dose of product is a machine for administering a radiopharmaceutical drug to a patient and the machine also comprises a disposable radiopharmaceutical kit with at least one syringe for administering the dose to the patient.

A fourth aspect of the invention relates to a method for the reversible coupling and separation of at least one syringe and of an actuator of the syringe, implementing the coupling device described hereinabove, comprising steps for coupling and separating, the coupling being characterized by the following steps:

the coupling device is in the rest position, with the coupling body protruding relative to the sleeve in the direction of the syringe, preferably upwards when the syringe is located above the coupling device, downwards respectively when the syringe is located below the coupling device;

the actuator pushes the coupling toward the pusher button of the syringe;

the pusher button pushes on the hooks at its chamfer and causes the hooks to open, the bulges of the hooks successively shifting from a contact with the hollow zone of a first depth of the internal profile of the sleeve to a non-contact position;

as soon as the pusher button reaches the end of the protruding part of the hooks, the hooks automatically close on the pusher button, due to the return spring, the pusher button and the play-take-up plate coming into contact and inserting into the recess; and

the pusher button is locked without play between the hooks and the play-take-up plate.

Advantageously, the method for reversibly coupling and separating a syringe and an actuator from the syringe, implementing the coupling device described hereinabove, has a separation characterized by the following steps, once the reversible coupling of the syringe and actuator is performed as described hereinabove:

from the rest position of the coupling, the actuator pulls the coupling in the retraction direction of the pusher, preferably downwards or upwards, respectively, depending on the relative position of the actuator and of the syringe, causing the pusher to retract in the syringe;

the sleeve abuts against a bearing surface of the actuator chassis and stops while the body continues to move, preferably downwards, respectively upwards, under the action of the actuator;

the outer profile of the hooks cooperates by contact with the hollow zone of the internal profile of the sleeve, the bulges of the hooks successively shifting from a contact with the hollow zone of a first depth of the internal profile of the sleeve to a contact with the hollow zone of a second depth of said profile, so as to pivot and open the hooks;

the pusher button separates from the coupling and, if a negative pressure has been created beforehand in the syringe by closing a valve communicating in a downstream line with the syringe, the pusher automatically penetrates the syringe body as soon as the hooks are opened;

the syringe is released from the coupling and may be removed.

Alternatively, the method for reversibly coupling and disconnecting a syringe and an actuator of the syringe, implementing the device described above, is characterized by the following steps (not included in the invention):

from the rest position of the coupling, the sleeve is manually moved toward the pusher of the syringe, preferably the sleeve is manually raised or lowered respectively toward the pusher of the syringe;

the outer profile of the hooks interacts by contact with the hollow internal profile of the sleeve so as to pivot and open the hooks, the bulges of the hooks successively shifting from a contact with the hollow zone of a first depth of the internal profile of the sleeve to a contact with the hollow zone of a second depth of said profile;

the pusher button disengages from the coupling and, if a vacuum has been created beforehand in the syringe by closing a valve communicating in a downstream line with the syringe, the pusher automatically enters the syringe body as soon as the hooks are opened, withdrawing the actuator alternatively allowing to disengage the pusher button from the coupling device;

the syringe is released from the coupling and can be removed or used in place to continue the collection and injection operations by manual movements of the pusher.

Thus, in the event of a machine failure, manual operation allows to release the pusher from the faulty actuator.

A specific system for gripping the pusher of the syringe has been developed according to the present invention.

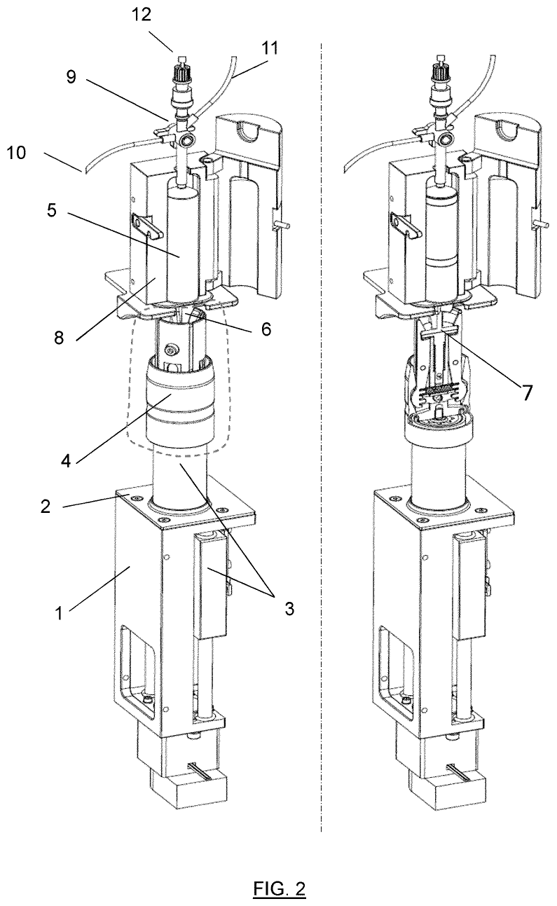

FIG. 2 shows a fixed structure 1 with its bearing surface 2 for the coupling, the syringe actuator 3, its coupling device 4, the syringe with its body 5 and pusher 6 in the position secured to the coupling 4, the system 8 for securing the syringe to a chassis, the three-way valve 9 and the beginning of the respective collection tube 10, saline connection tube 11 and injection tube 12.

On the right-hand side of FIG. 2, the coupling 4 is detailed in a sectional view. The pusher button 7 of the syringe inserted into the coupling can also be seen.

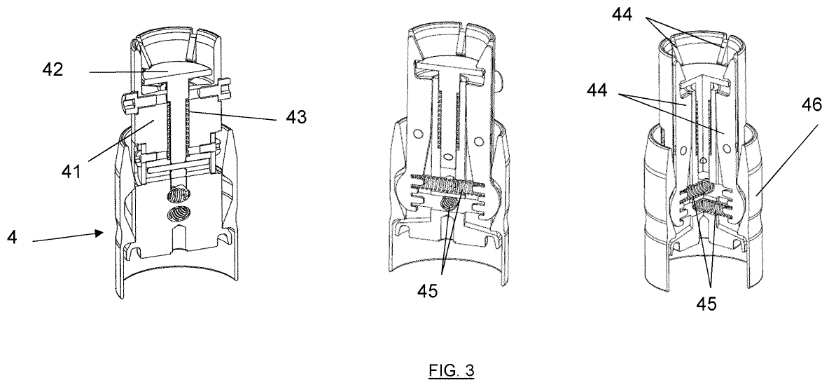

FIG. 3 shows the passive coupling device 4 alone with its different components, namely:

a coupling body 41;

a play-take-up plate 42 and its prestressed spring 43;

a plurality of pivoting hooks 44;

a prestressed return spring 45 for each pair of hooks 44; and

a sleeve 46 for controlling the opening of the hooks 44.

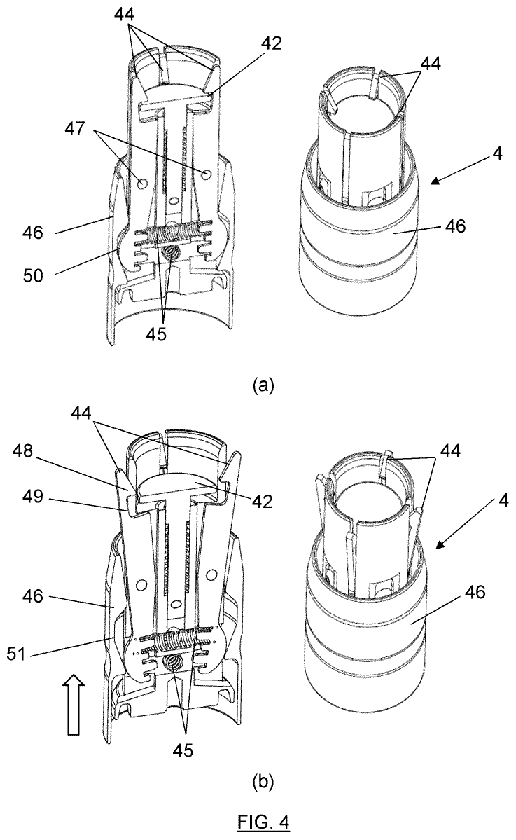

FIG. 4a) shows the coupling 4 in the default (or rest) state, namely with closed hooks 44. FIG. 4b) shows the coupling 4 with open hooks 44 due to the relative movement of the sleeve 46 with respect to the body of the coupling 41.

According to one embodiment, the hooks 44 are longitudinally mounted in pairs, the hooks of a same pair being connected by a return spring 45 arranged along an axis perpendicular to the mean axis of the hooks and secured to the hooks in the lower part thereof. There are e.g. two perpendicular pairs 44 of hooks, the respective springs 45 thereof also being perpendicular and situated at different heights. The hooks 44 are mounted so as to pivot about a pin 47 located approximately at half the height of the hook. In the top-down direction, each hook 44 has an inwardly protruding part 48 followed by a recess 49, which will allow to lock the button pusher 7 of the syringe against the play-take-up plate 42, once they are joined. Furthermore, the raising movement of the sleeve 46 with respect to the coupling body 41 will serve, according to the invention, to open the hooks and to release the pusher button 7 and the pusher 6 of the syringe.

To this end, the external structure of the hooks 44 must cooperate with the internal structure of the sleeve 46. According to one embodiment, the lower outer part of the hooks is provided with a bulge 50 and the internal part of the sleeve is provided with a hollow profile 51 corresponding to the desired displacement of the bulge 50 when the sleeve rises relative to the body of the coupling. In the default position, the hooks 44 of the coupling 4 being closed, the bulge 50 of the hooks is located in the hollow of the profile 51 of the sleeve (FIG. 4a). When the coupling body 41 moves down relative to the sleeve, the bulge 50 exits downwards from the internal profile of the sleeve 51 and the contact of the bulge 50 with the lower edge of the protruding profile 51 automatically pivots each hook 44 about its pivot pin 47, so that the hook opens outwards and releases the pusher button 7 and consequently the pusher 6 of the syringe.

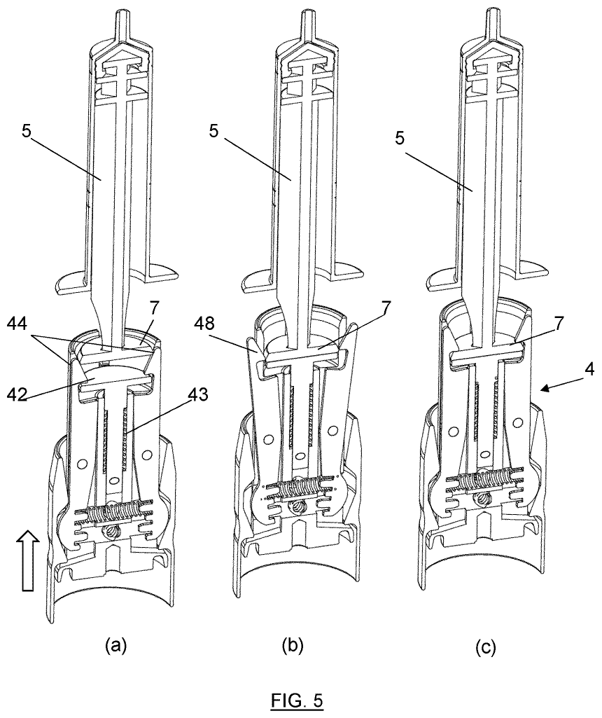

FIG. 5 shows in three successive steps the mechanism for securing the syringe 5 (assumed to be fixed) with relative movement of the coupling 4 with respect to the syringe 5 and opening/closing of the hooks 44.

By moving along the injection direction, the coupling 4 comes into contact with the button pusher 7 of the syringe 5 and hooks thereto. The opening of the hooks 44 is caused by the contact of the pusher button 7 of the syringe 5 on the hooks 44 (FIG. 5b). The closure of the hooks 44 on the button pusher 7 occurs as soon as the pusher button 7 of the syringe 5 sticks out beyond the end of the protruding parts 48 of the hooks 24 (FIG. 5b), thereby compressing the play-take-up plate 42 mounted on the prestressed spring 43 so as to be embedded into the recess 49 of the hooks (FIG. 5c).

The plate 42 mounted on its prestressed spring 43 takes up the plays between the gripping hooks 44 and the upper bearing surface of the button 7 of the pusher and between the lower bearing surface of the pusher button 7 and the play-take-up plate 42. The hooking process requires overcoming the force of the spring.

Once hooked together, the pusher 6 and the actuator 3 are strictly held together whatever the motion direction of the actuator 3 (FIG. 5c) within the limit of the force of the prestressed spring of the plate.

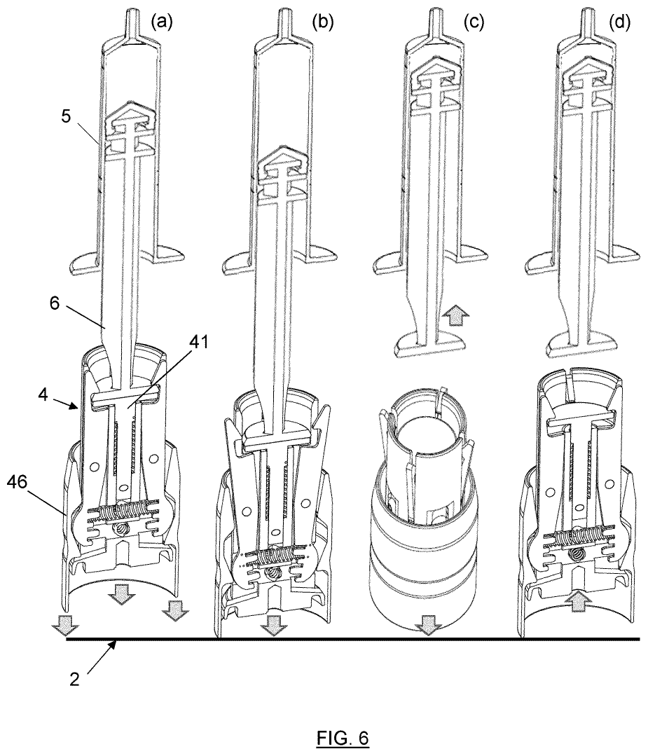

FIG. 6 then shows the different steps for separating the syringe (from left to right). Separation is activated by a maximum downward retraction motion of the actuator (FIG. 6a). When the coupling 4 is lowered, the sleeve 46 comes into contact with the bearing surface 2 of the fixed structure 1 which prevents the sleeve 46 from moving further down, while the body 41 of the coupling 4 continues to move down. By its relative movement with respect to the body of the coupling 41, the sleeve 46 uses its internal profile to bear on the bulges of the hooks 44 so as to pivot and open them (as described hereinabove). As the hooks move apart, the pusher button 7 then is detached from the coupling 4 and the pusher 6 can automatically rise upwards. Indeed, if the downward motion of the pusher 6 has been achieved with the three-way valve in the closed position, it causes a negative pressure in the syringe and the pusher 6 instantaneously rises back into the body 5 of the syringe as soon as the hooks 44 are opened. Syringe 5 is released from coupling 4 and the kit can easily be removed from the machine.

The uncoupling can also be carried out manually by lifting the sleeve 46, the actuator being stopped (does not belong to the invention).

The sleeve 46 may be attached either (a) to the coupling (as shown above) or (b) to the bearing surface of the fixed structure. It should be noticed that, in the latter case, it could not be actuated manually.

### DESCRIPTION OF PREFERRED EMBODIMENTS OF THE INVENTION

In a preferred embodiment of the invention, the syringe has a nominal capacity of 10 ml for a stroke on the order of 48 mm, with the plunger having the possibility to move by several mm beyond the nominal filling. 10 to 15 ml represents a typical volume of radiopharmaceutical delivered in vials for PET (positron emission tomography).

A pediatric dose of 18F-FDG e.g. can be as small as 30 MBq, whereas an adult dose is in the range of 200 to 300 MBq. If the concentration of the starting solution is high, e.g. 30 GBq in 10 ml, and a pediatric dose should be prepared, it is the most difficult case. The volume to be withdrawn is then 10*(30/30000) ml=0.01 ml (=10 μl).

The smallest collection considered, and thus the smallest variation of volume (ΔV) targeted, is ˜0.0 1mL with an expected precision of ±10%. Such ΔV corresponds to a displacement of 0.01 [ml]*(48 mm/10 ml)=0.048 mm, i.e. 48±6 μm.

The resolution of the push-syringe device being ˜2 μm, such dosages are only possible with the desired precision in the absence of any play and with good control of the applied forces.

According to another embodiment of the invention, a force sensor is arranged behind the coupling mechanism between the actuator and the coupling device and serves to directly measure the forces during injection and during coupling, which allows in particular:

by producing vacuum in the syringe, to check that the coupling has indeed been carried out;

to check the pressure differences in the kit because the pressure exerted by the substance in the syringe on the syringe plunger is transmitted to the pusher button as such directly connected to the coupling, which is itself connected to the actuator via the force sensor in question. Anomalies such as a crushed tube in the kit can thereby be detected when fluid is moved with the syringe.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SYMBOLS

1 fixed structure (chassis)
2 bearing surface
3 actuator
4 coupling
5 syringe (barrel)
6 pusher
7 pusher button
8 securement system of the syringe
9 three-way valve
10 collection line
11 saline connection line
12 injection line
41 coupling body
42 play-take-up plate
43 pre-stressed spring (from the take-up plate)
44 pivoting hook
45 return spring (pair of hooks)
46 coupling sleeve
47 pivot pin
48 protruding part of the hook
49 recess of the hook
50 bulge
51 internal profile of the sleeve
100 radiopharmaceutical kit
101 syringe
102 valve-syringe connection line
103 three-way valve
104 T-shaped tube
105, 105′ non-return valve
106 collection line
107 vial of concentrated radiopharmaceutical
108 saline connection line
109 saline vial
110 injection line
111 ventilated filter
112, 112′ bubble detector
113 self-closing Luer
114 patient extension set
115 machine Luer fitting
116 patient Luer fitting
117 dose calibrator
118 piercing needle

1. A system for reversibly coupling a syringe for injecting a calibrated product to an actuator comprising the actuator, the syringe provided with a pusher having a pusher button and a coupling device, the coupling device comprising an internal body with longitudinal axis and an outer sleeve intended to move with respect to the internal body along the longitudinal axis, the internal body comprising a sliding pin ending with a play-take-up plate that is held longitudinally inside the internal body in a rest position by a prestressed spring as well as at least one pair of hooks arranged longitudinally inside the internal body mounted pivoting and retractable two by two with respect to two respective pins perpendicular to the longitudinal axis, an external part of the hooks being located opposite an internal part of the sleeve, the hooks of a same pair each having a zone for hooking to a proximal end of the play-take-up plate, and being connected to each other to a distal end by a return spring,
wherein the distal part of the hooks is provided, outwards, with a bulge intended to be inserted into a hollow zone of a first depth of the internal profile of the sleeve in a rest position of the coupling device where the hooks is are upright and in position for horizontally locking the pusher button with the play-take-up plate,
wherein—the relative sliding of the sleeve with respect to the internal body, wherein the sleeve moves away from the distal end of the hooks causes the hooks to open by pivoting about their respective pivoting pins, because the bulge of each hook penetrates in the direction opposite that of the motion of the sleeve, a hollow zone of a second depth of the internal profile of the sleeve the hollow zone of a second depth being adjacent to the hollow zone of a first depth, the first depth being greater than the second depth, and
wherein the region for hooking the hook is defined by a protruding portion with an inwardly sloping chamfer followed by a recess below the protruding portion, the recess being dimensioned so as to lock by means of the hooks, the play-take-up plate adjoining the pusher button of the syringe, the play-take-up plate exerting, thanks to the prestressed spring, a force directed toward the pusher button so as to eliminate any play between the pusher button and the coupling device.

2. The reversible coupling system of claim 1, wherein the coupling device comprises from 2 to 4 pairs of hooks.

3. (canceled)

4. (canceled)

5. The reversible coupling system of claim 41, further comprising:
a force sensor arranged behind the coupling mechanism between the actuator and the coupling device and intended to directly measure the forces during the injection and during the coupling.

6. An automatic machine for administering a dose of product, comprising:
the reversible coupling system of the syringe and actuator of claim 41.

7. The automatic machine for administering a dose of product of claim wherein the product is comprises a radiopharmaceutical drug, and
wherein the machine comprises a disposable radiopharmaceutical kit comprising at least one syringe for the administration of the dose to the patient.

8. A method for the reversible coupling and separation of at least one syringe and of an actuator from the syringe implementing the coupling device of claim 1, comprising coupling steps and separation steps, the coupling comprising:
the coupling device is in the rest position, with the coupling body protruding relative to the sleeve toward the syringe;
the actuator pushes the coupling toward the pusher button of the syringe;
the pusher button pushes on the hooks at the level of its chamfer and causes the hooks to open, the bulges of the hooks successively shifting from a contact with the hollow zone of a first depth of the internal profile of the sleeve to a non-contact position;
as soon as the pusher button has reached the end of the protruding part of the hooks, the closing of the hooks on the pusher button is automatic, due to the return spring, the pusher button and the play-take-up plate coming into contact and inserting into the recess; and
the pusher button is locked without play between the hooks and the play-take-up plate

9. The method for the reversible coupling and separation of a syringe and of an actuator from the syringe, the separation comprising, once the reversible coupling of the syringe and of the actuator of claim 8 is performed:
from the rest position of the coupling, the actuator pulls the coupling along the direction for withdrawing the pusher from the syringe;
the sleeve abuts against a bearing surface of the chassis of the actuator and stops while the body continues to move under the effect of the actuator;
the outer profile of the hooks cooperates by contact with the hollow zone of the internal profile of the sleeve the bulges of the hooks successively shifting from a contact with the hollow zone of a first depth of the internal profile of the sleeve to a contact with the hollow zone of a second depth of the profile so as to pivot and open the hooks;
the pusher button separates from the coupling and, if a negative pressure has been created beforehand in the syringe by closing a valve communicating in a downstream line with the syringe, the pusher automatically rises into the syringe body as soon as the hooks are opened; and
the syringe is released from the coupling and may be removed.
